(19) United States
(12) Patent Application Publication
(10) Pub. No.: US 2007/0089744 A1
(43) Pub. Date: Apr. 26, 2007

(54) Method for determining a cardiac data characteristic

(76) Inventor: Scott R. Wiese, Milwaukee, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/247,616

(22) Filed: Oct. 11, 2005

(51) Int. Cl. A61M 16/00; A61B 5/08

(52) U.S. Cl. 128/204.23; 600/529

(57) ABSTRACT

A method for determining a blood pressure characteristic, such as SPV and/or PPV of a patient supplied with breathing gases from a mechanical ventilator does not require breathing gas waveforms. An amount of blood pressure data that is at least substantially equal in time to the duration of a breath of the patient is selected. The selection of the amount is not limited to one defined by the commencement or conclusion of a breath of the patient. Preferably blood pressure data in an amount corresponding to the unit of time used to express a respiratory rate is employed and the amount is divided into segments equal in number to the breath number of the respiratory rate. Each of the segments will contain data from a respiratory cycle of the patient although the data for one of the inspiratory/expiratory phases of the respiratory cycle may comprise complementary portions from two successive inspiratory or expiratory phases included in the segment. The blood pressure characteristic is determined from the selected blood pressure data amount.

### BACKGROUND AND SUMMARY

The present invention relates to a method for determining a cardiac data characteristic that is influenced by a patient's respiration. The medical data characteristic may comprise the variation in systolic blood pressure (SPV) and/or the variation in pulse pressure (PPV) due to respiration by the patient.

Systolic blood pressure represents the maximum force exerted by the blood within the blood vessels of the circulatory system. This occurs when the heart contracts during a heartbeat. Pulse pressure is the difference between the systolic blood pressure and the diastolic blood pressure. The diastolic blood pressure is the minimum force of the blood within the blood vessels experienced when the heart relaxes. It is often desirable to frequently measure the variation in systolic pressure and the variation in pulse pressure while the patient is receiving certain forms of medical care or treatment especially when the treatment involves fluid therapy since changes in SPV and PPV are a clinically accepted way to ascertain the state of intravascular fluid volume.

Systolic blood pressure and pulse pressure may be measured using a variety of techniques, but direct measurement of blood pressure using invasive arterial blood pressure is often preferred in critical care situations. This is because of the high frequency response of this technique as well as its accuracy and the elimination of time delays experienced when using other methods.

In the ventilation of a patient with a mechanical ventilator, breathing gases are supplied to the patient under positive gas pressure via a breathing circuit during inspiration. The breathing gases are supplied at a pressure sufficient to overcome resistance of the breathing circuit and the patient's airway, lungs, and chest wall, resulting in the inspiration of the breathing gases. In the expiration phase of each breathing cycle, the pressure is released and the natural relaxation of the patient's thorax and the compliance of the patient's chest wall discharges breathing gases from the lungs.

The pressure generated in the chest during inspiration is transmitted to the thoracic structures of the body, including the heart, and the great arteries and veins of the circulatory system. This increased intrathoracic pressure results in a decrease in systolic and diastolic pressures. This is often termed the “respiratory swing” and is more pronounced under conditions of reduced blood volume, known as “hypovolemia”, so that changes in the magnitude of the respiratory swing, as reflected in SPV and PPV, may be used to detect changes in patient blood volume.

Current methods of determining SPV and PPV require the concurrent measurement of the patient's airway waveform to correlate the blood pressure measurement to the respiratory cycles. The calculation techniques employ breathing gas waveforms, such as airway pressures or CO₂ amounts, to determine when respiratory cycles begin and end so that the blood pressure measurement may be divided into segments corresponding to the respiratory cycles. A patient monitoring system receives these complex, continuous breathing gas waveforms via a data connection between an airway pressure sensor, or gas analyzer, and the monitoring system. Additionally, the patient monitoring system typically also receives data, usually in the form of periodic “packets” of medical data, from other equipment, such as the mechanical ventilator or other sensors to which the system is also connected. These packets provide a periodic update of patient or apparatus parameters for use in determining and displaying medical data quantities and/or controlling medical apparatus.

In some clinical settings such as an operating room or intensive care unit, however, the patient monitoring system cannot properly communicate with the ventilator or breathing gas sensor(s) being used in order to receive the breathing gas waveform data necessary to segment the blood pressure measurement into respiratory cycles. This situation may arise if the ventilator has not been properly interfaced with the patient monitoring system. An improper interface may result from a faulty data connection, or because the pieces of equipment were designed by different manufacturers thus resulting in limitations on compatibility. Also, in situations where multiple pieces of equipment are connected to a patient monitoring system, data transfer from some pieces of equipment may take priority over others and may block the receipt of some information by the patient monitoring system. If any of these situations arises, it may result in the patient monitoring system not receiving the patient breathing gas waveform data in a complete and timely manner. In these situations, the patient monitoring system is often able to only receive limited data packets which are not robust enough or frequent enough for current methods of determining SPV and PPV to be performed.

Therefore it is desirable to provide a method for determining SPV and PPV, as well as other respiratory dependent characteristics, based upon respiratory information that is less complex or can be received less frequently than the heretofore used breathing gas waveform data. This can provide a redundant or backup measurement of SPV and PPV while a patient is mechanically ventilated to ensure that SPV and PPV data quantities will be available to the clinician, even in the event that patient breathing gas waveform data is not available to the patient monitoring system or is inadequate.

### BRIEF DESCRIPTION OF THE INVENTION

In an exemplary aspect of the invention, a signal representative of respiratory rate, rather than breathing gas waveform data, is used in the determination of cardiac data characteristics, such as SPV and PPV. A unique insight employed in the present invention is that breathing gas pressure or gas composition waveform data is not necessary to determine SPV or PPV but rather the determination of SPV or PPV may be carried out using only respiratory rate data received from a ventilator or other source. Respiratory rate, usually a digit indicative of the number of breaths per minute, is far less complex in data content than the breathing gas waveform data used in the past. The respiratory rate can be simply and definitively provided to the patient monitor, even when the monitor is connected to other equipment thus ensuring its availability when needed for use in determining SPV and/or PPV.

In a conveniently understood embodiment of the invention, a continuum of blood pressure data is obtained from the patient over a period of time. The mechanical ventilator for the patient typically supplies breathing gases at a respiratory rate expressed as a number of breaths per unit time, for example 12 breaths per minute. A selection of a blood pressure data amount is then made. It is convenient to select an extent in time for the blood pressure data equal to the unit of time used to express the respiratory rate, i.e. blood pressure data occurring in one minute. In contrast to techniques using breathing gas waveforms, with the present invention, the selection of the blood pressure data is not limited to one defined by the commencement and conclusion of a breath of the patient.

The selected blood pressure data is then divided into segments equal in number to the breath number in the respiratory rate, in the present example, twelve segments of 1/12 of a minute each. Blood pressure data from one or more of the segments is then used to compute the medical data characteristic, such as SPV and/or PPV.

It will be appreciated that the blood pressure data segments will contain data from a respiratory cycle of the patient but that the beginning and end of a blood pressure data segment used to compute SPV and/or PPV may not correspond to the beginning of an inspiratory phase of the patient's respiratory cycle and the end of an expiratory phase, respectively. However, since the extent of the blood pressure data in a segment is that of the duration of the patient's respiratory cycle, the data of a blood pressure segment will contain that of an inspiratory phase and an expiratory phase, although the data for one of the phases may comprise complementary portions from two successive inspiratory or expiratory phases included in the segment. This enables the medical data characteristic to be determined.

Thus, with the present invention, it is not necessary to know the rather considerable breathing gas pressure or gas analysis waveform information used in the past, but only the simple respiratory rate established by the mechanical ventilator, which information can be easily and definitively provided by the ventilator, a pressure sensor in the breathing circuit, or a gas analyzer in the breathing circuit. The medical data characteristic can be correspondingly easily and definitely provided.

### BRIEF DESCRIPTION OF THE DRAWINGS

FIG. 1 shows a mechanical ventilation and monitoring arrangement for a patient;

FIG. 2 shows a blood pressure responsive catheter for a patient;

FIG. 3 shows blood pressure and airway pressure waveforms for a mechanically ventilated patient; and

FIG. 4 is a flow chart showing the method of the present invention.

### DETAILED DESCRIPTION

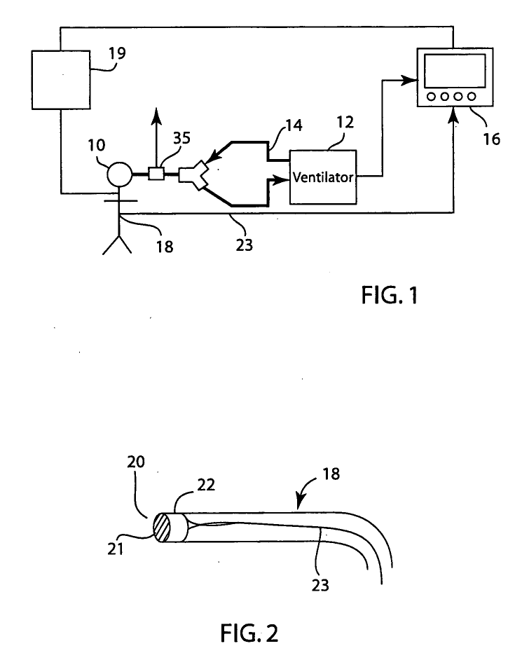

FIG. 1 depicts patient 10 connected to mechanical ventilator 12, breathing circuit 14, and patient monitor 16. Monitor 16 obtains blood pressure data from the patient 10. For this purpose, catheter 18 may be inserted in the circulatory system of patient 10. The patient's brachial artery is often used, but it is understood that arterial blood pressure may be taken from any major artery, including the femoral artery or radial artery.

Monitor 16 may also be connected to other monitoring or treatment equipment for patient 10, shown in FIG. 1 as 19.

FIG. 2 depicts a catheter 18 capable of transducing invasive arterial blood pressure. The catheter 18 comprises a tip 20 with a diaphragm 21 and a sensor 22. It is understood that a number of different types of sensors may be used in catheter 18 including a strain gauge, linear-variable differential transformer, variable inductance, variable capacitance, piezoelectric, or semi conductor devices. The signal from the catheter is sent via conductor 23 to monitor 16 for analysis and/or display.

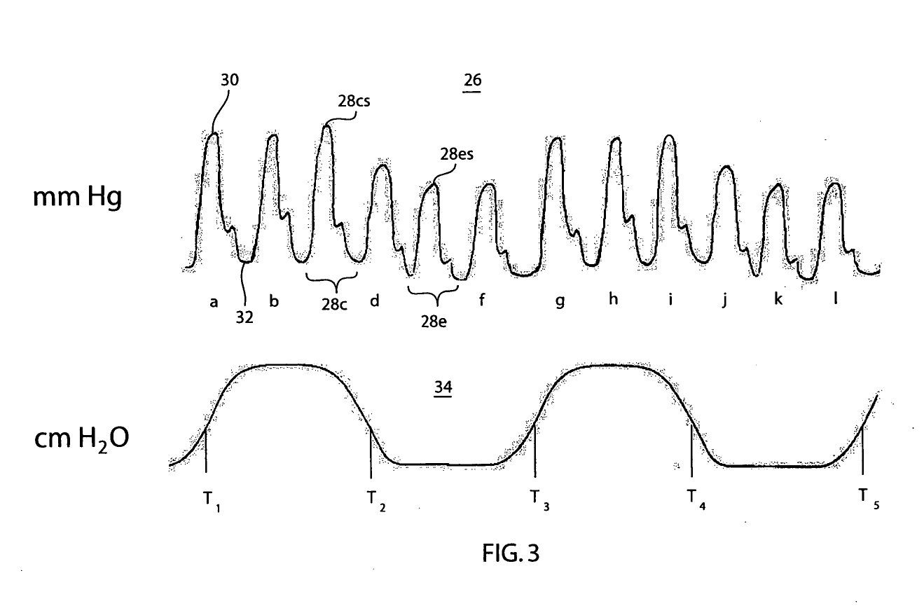

FIG. 3 depicts graphs representative of arterial blood pressure and airway pressure waveforms. Graph 26 is representative of an arterial pressure waveform in units of mmHg. The arterial pressure waveform 26 shows a series of pressure waves 28a-l each representative of the arterial blood pressure over a heartbeat or pulse cycle. A typical heartbeat rate is 60 to 100 beats per minute. Each pulse waveform has a peak or systolic pressure 30 and a minimum or diastolic pressure 32.

Graph 34 is representative of the pressure in the patient's airway as established by ventilator 12 or obtained from sensor 35 located in breathing circuit 14 extending between patient 10 and mechanical ventilator 12. Airway pressure is typically expressed in units of cmH₂O and is indicative of the intrathoracic pressure existing in the patient. A respiratory cycle for patient 10 comprises an inspiratory phase occurring, for example, between time T1 and T2 and time T3 and T4 during which ventilator 12 provides pressurized breathing gases to patient 10 and an expiratory phase occurring, for example, between times T2 and T3 and time T4 and T5 during which patient 10 expires breathing gases. A full respiratory cycle thus comprises time T1 to T3 and time T3 to T5. The pressure in breathing circuit 14 may drop to ambient at the end of a respiratory cycle. Or, a small positive pressure, termed “positive end expiratory pressure” (PEEP) may be maintained by ventilator 12 at the end of the respiratory cycles. A typical respiratory rate is 12 to 22 breaths per minute.

Other techniques in addition to airway pressure may be used to identify the respiratory cycles of patient 10. For example, sensor 35 may comprise a CO₂ gas sensor connected in the breathing circuit. The amount of CO₂ in the expired breathing gases of patient 10 reaches a peak at the end of expiration so that the respiratory cycles may be defined as the interval between CO₂ peaks.

FIG. 3 shows the respiratory swing in the blood pressure data over a respiratory cycle of patient 10 in which, particularly, the systolic blood pressures are reduced in time interval T₂-T₃ over those seen in time interval T₁-T₂ as a result of the increased thoracic pressures from mechanical ventilation during inspiration. In FIG. 3, blood pressure waves 28a-f correspond to the first respiratory cycle T1-T3 of the patient. The systolic pressure variation (SPV), is the difference between the highest systolic pressure in the series, depicted as blood pressure wave 28cs, and the lowest systolic blood pressure, depicted as pressure wave 28es. The pulse pressure is determined within a single cardiac cycle by subtracting the diastolic pressure 32 from the systolic pressure 30. The pulse pressure variation (PPV) is determined as the greatest difference between the systolic and diastolic pulse pressures found in a cycle of the series of cardiac cycles of a respiratory cycle.

The blood pressure waveform 26 need to be related to the respiratory cycles in order to determine SPV and PPV. As depicted in FIG. 3, cardiac cycles 28a-f are associated with the first respiratory cycle T1-T3, and cardiac cycles 28g-l are associated with the second respiratory cycle T3-T5. As seen from the figure, cycle 28c showing the maximum systolic pressure, and pulse pressure variation is correlated with the inspiratory portion T1-T2 of signal 34 indicative of the patient's respiratory cycles. The cycles expressing minimum systolic blood pressure and the minimum pulse pressure variation, such as 28e, are correlated with the expiratory portions T2-T3 and T4-T5 of the patient's respiratory cycles.

Prior art methods of calculating SPV and PPV have been dependent on correlating the respiratory cycles, as exemplarily embodied in airway pressure data 34 measured by sensor 35, with the blood pressure data 26 obtained from the patient as by catheter 18, in order to divide the arterial blood pressure data into segments, 28a-f; 28g-l representative of the respiratory cycles T1-T3; T3-T5, respectively, of the patient. As noted above it is not always possible to transmit the considerable amount of airway pressure or exhaled breathing gas data to the patient monitor 16 so that the determination of SPV and PPV may be lost or rendered inaccurate.

The present invention, however, recognizes that as the determination of SPV and PPV is only made when a patient is being mechanically ventilated, the respiratory cycles will be those established by the mechanical ventilator. The particular phase of the respiration cycle is not needed when the patient is mechanically ventilated because any segment of blood pressure data of substantially the same, or greater, time duration as that of a breath established by the mechanical ventilator respiration rate will include one maximum systolic blood pressure and one minimum systolic blood pressure as well as the necessary systolic-diastolic cardiac cycle information, thereby enabling accurate determination of the SPV and PPV characteristics to be carried out. Further as the respiration rate is ordinarily held constant among successive respiratory cycles during mechanical ventilation, a single quantity representing respiratory rate of the mechanical ventilator may be used to define the respiratory cycles for a given time series or continuous of blood pressure data. Thus only the respiration rate needs to be provided to monitor 16. As the respiration rate is a small amount of data, such as a single digit, it can be easily transmitted to the monitor to ensure that the determination of SPV and PPV can be made.

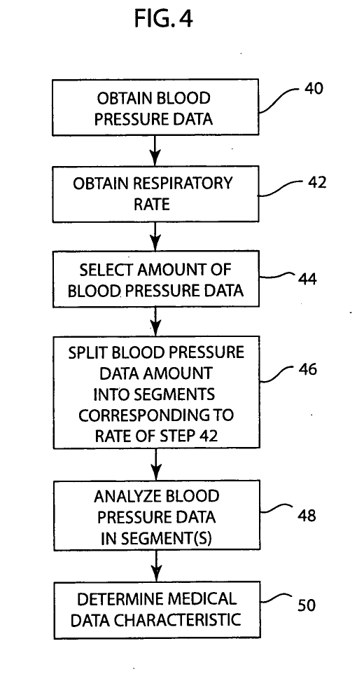

FIG. 4 is a flowchart representing the steps of an embodiment of a method for determining cardiac data characteristics, such as SPV and PPV. The method may be carried out by an appropriately programmed microprocessor in monitor 16. In step 40, the blood pressure data 26 shown in FIG. 3 is obtained. The respiratory rate of patient 10 is also obtained, preferably from ventilator 12. Respiratory rate data may also be obtained from a sensor 35, such as a gas analyzer in the breathing circuit. Or, it may be manually entered by a clinician into monitor 16. Obtaining the respiratory rate is carried out in step 42.

An amount of blood pressure data is selected. See step 44. At a minimum, the extent of the selected amount generally corresponds to the duration of the breaths of the patient. Importantly, selection of the amount of blood pressure data is not limited to that defined by the commencement and conclusion of a breath of a patient. The strict correlation of blood pressure and respiratory cycle data employed in past techniques is not required. In an embodiment of the invention, an amount of blood pressure data comprising that for the period of time used to define the respiratory rate may be selected, i.e., a one minute amount of data is obtained. From the selected amount of blood pressure data, at least one segment equal to a fractional amount defined by one over the breath number in the respiratory rate. Thus, if the respiratory rate is twelve breaths per minute, the segment would be 1/12^{th} of the selected amount. In a preferred embodiment of the invention, the obtained data is divided into a plurality of segments equal in number to the numerator, or breath number, in the respiratory rate for patient 10. Thus if the respiratory rate is twelve breaths per minute, a one minute amount of blood pressure data is obtained and divided into 12 segments. See step 46. Each of the segments will thus have a duration generally equal to that of a respiratory cycle of patient 10. Again, as noted above, the segment or segments need not correlate with the beginning and end of the respiratory cycles.

The blood pressure data from one or more of the segments is then analyzed, in step 50, to obtain the systolic and diastolic pressures necessary to determine SPV and PPV medical data characteristics in the manner described above in connection with FIG. 3. Since the segments are of a duration generally corresponding to the duration of a respiratory cycle, the segments will contain the data for a respiratory swing of the arterial blood pressure data enabling these characteristics to be accurately determined. Determined SPV and PPV blood pressure characteristics may be used to ascertain the intravascular fluid volume status of patient 10.

It is recognized that other equivalents, alternatives, and modifications in addition to those described above are possible and within the scope of the following claims.

What is claimed is:

1. A method for determining a blood pressure characteristic of a patient supplied with breathing gases from a mechanical ventilator, the mechanical ventilator supplying the breathing gases in breaths, each of which has a duration, said method comprising the steps of:
obtaining a continuum of blood pressure data from the patient, the blood pressure data being periodically alterable by breathing of the patient;
selecting, from the blood pressure data, an amount that is at least substantially equal in time to the duration of a breath of the patient, the selection of the amount not being limited to one defined by the commencement and conclusion of a breath of the patient; and
determining the blood pressure characteristic from the data of the selected amount.

2. The method of claim 1 wherein the mechanical ventilator supplies breathing gases to the patient at a respiratory rate expressed as a number of breaths per unit time, said method including the steps of:
selecting an extent in time for the blood pressure data amount corresponding to the unit of time used to express the respiratory rate;
obtaining a segment of the blood pressure data amount comprising that fraction of the amount represented by one over the breath number in the respiratory rate; and
the determining step is further defined as determining the blood pressure characteristic from the data of the segment of the portion.

3. The method of claim 2 wherein the segment obtaining step is further defined as dividing the blood pressure data amount into segments equal in number to the breath number of the respiratory rate and the determining step is further defined as determining the blood pressure characteristic from one or more of the segments.

4. The method of claim 1 wherein the blood pressure data is arterial blood pressure data.

5. The method of claim 1 wherein the blood pressure data includes systolic and diastolic blood pressure data and wherein the determined blood pressure characteristic is at least one of systolic pressure variation (SPV) and pulse pressure variation (PPV).

6. The method of claim 2 further including the step of obtaining an indication of the patient respiratory rate from the mechanical ventilator.

7. The method of claim 2 further including the step of obtaining an indication of the patient respiratory rate from a breathing gas sensor.

8. The method of claim 2 further including the step of obtaining the patient respiratory rate as a clinician entered respiratory rate.

9. The method of claim 1 wherein the blood pressure data comprises invasively obtained blood pressure data.

10. A method for determining a blood pressure characteristic of a patient supplied with breathing gases from a mechanical ventilator, the mechanical ventilator supplying the breathing gases at a respiratory rate expressed or a number of breaths per unit time, said method comprising the steps of:
obtaining a continuum of blood pressure data from the patient, the blood pressure data being periodically alterable by breathing of the patient, the blood pressure data including systolic and diastolic blood pressure data;
selecting, from the obtained blood pressure data, a portion having an extent in time corresponding to the unit of time used to express the respiratory rate, the selection of the portion not being limited to one defined by the commencement and conclusion of a breath of the patient; and
dividing the blood pressure data portion into segments equal in number to the breath number in the respiratory rate; and
determining at least one of the systolic pressure variation (SPV) and pulse pressure variation (PPV) from at least one of the segments.
